(19) United States
(12) Patent Application Publication
(10) Pub. No.: US 2020/0019823 A1
(43) Pub. Date: Jan. 16, 2020

(54) MEDICAL IMAGE ANALYSIS METHOD APPLYING MACHINE LEARNING AND SYSTEM THEREOF

(71) Applicant: National Taiwan University of Science and Technology, Taipei (TW)

(72) Inventor: Ching-Wei Wang, Taipei (TW)

(73) Assignee: National Taiwan University of Science and Technology, Taipei (TW)

(21) Appl. No.: 16/111,216

(22) Filed: Aug. 24, 2018

(30) Foreign Application Priority Data
Jul. 12, 2018 (TW) 107124061

(51) Int. Cl. G06K 9/62; G06N 3/08; G06N 3/04; G16H 30/20

(52) U.S. Cl. CPC G06K 9/6262; G06N 3/08; G06N 3/04; G06K 2209/05; G06K 9/6256; G06K 9/6254; G16H 30/20

(57) ABSTRACT

A medical image analysis method applying machine learning and system thereof are provided. The medical image analysis system includes a cloud server and an electronic device. The cloud server stores a deep learning module and an artificial intelligence model. The medical image analysis method includes the following steps. Correction data is inputted to the deep learning module so that the deep learning module corrects the artificial intelligence model according to the correction data to generate a corrected artificial intelligence model. Furthermore, medical image data is inputted to the electronic device, and the electronic device provides the medical image data to the cloud server to analyze the medical image data through the corrected artificial intelligence model and generates analysis result data.

### CROSS-REFERENCE TO RELATED APPLICATION

This application claims the priority benefit of Taiwan application serial no. 107124061, filed on Jul. 12, 2018. The entirety of the above-mentioned patent application is hereby incorporated by reference herein and made a part of this specification.

### BACKGROUND

### Technical Field

The invention relates to an analysis method applying machine learning. More particularly, the invention relates to a medical image analysis method applying machine learning and system thereof.

### Description of Related Art

Cancer has become one of the leading causes of death in developing countries. In the diagnosis of cancer, the pathology section of the examination reports is one of the most important diagnostic criteria. In general, a doctor can judge the growth speed of the tumor, the grade of the disease, and the characteristics of the tumor through the pathological section of the examination reports, thus the pathological section of the examination reports plays an important role during the treatment of a patient. However, in a digital medical image, the medical image may have hundreds of millions of pixels. In other words, such large volume of data cannot be completely and accurately interpreted in a limited time. Moreover, when the medical images of the same pathological section are judged by different professionally-trained pathologists, a consensus of the pathological analysis among the pathologists is not high. Therefore, to effectively analyze the medical image data is an important issue, and solutions to this issue are provided in the embodiments below.

### SUMMARY

The invention provides a medical image analysis method and a system thereof using machine learning, which can generate artificial intelligence models through enhanced learning and continuous optimization, and the artificial intelligence model can effectively analyze the medical images.

A medical image analysis method using machine learning in an embodiment of the invention is adapted to a medical image analysis system. The medical image analysis system includes a cloud server and an electronic device. The cloud server stores a deep learning module and an artificial intelligence model. The medical image analysis method includes the following steps. Correction data is inputted to the deep learning module so that the deep learning module corrects the artificial intelligence model according to the correction data to generate a corrected artificial intelligence model. Furthermore, medical image data is inputted to the electronic device, and the electronic device provides the medical image data to the cloud server to analyze the medical image data through the corrected artificial intelligence model and generates analysis result data.

In an embodiment of the invention, the medical image analysis method further includes the following steps. Next correction data is generated according to the analysis result data, and the next correction data is inputted to the deep learning module. The corrected artificial intelligence model is corrected through the deep learning module according to the next correction data to generate a next corrected artificial intelligence model.

In an embodiment of the invention, the medical image analysis method further includes the following steps. Training data is inputted to the deep learning module so that the deep learning module builds the artificial intelligence model according to the training data.

In an embodiment of the invention, the training data includes a plurality of medical reference images, and the deep learning module includes a fully convolutional network module. The step of inputting the training data to the deep learning module so that the deep learning module builds the artificial intelligence model according to the training data includes the following steps: The fully convolutional network module is executed through the deep learning module so that the fully convolutional network module performs neural network operation on each of the medical reference images to build the artificial intelligence model.

In an embodiment of the invention, the fully convolutional network module performs upsampling operation on each of the medical reference images.

In an embodiment of the invention, the medical image analysis method further includes the following steps. Another medical image data is inputted to the electronic device, and the electronic device provides the other medical image data to the cloud server to analyze the other medical image data through the artificial intelligence model and generates another analysis result data. The correction data is generated according to the other analysis result data.

In an embodiment of the invention, the step of inputting the correction data to the deep learning module so that the deep learning module corrects the artificial intelligence model according to the correction data to generate the corrected artificial intelligence model includes the following steps. Another training data is further inputted to the deep learning module so that the deep learning module corrects the artificial intelligence model according to the correction data and the other training data to generate the corrected artificial intelligence model.

In an embodiment of the invention, a weight of the correction data is greater than a weight of the other training data.

In an embodiment of the invention, the other training data includes another medical reference image.

The medical image analysis system applying machine learning in an embodiment of the invention includes a cloud server and an electronic device. The cloud server stores a deep learning module and an artificial intelligence model. The electronic device is coupled to the cloud server. When the cloud server receives correction data, the deep learning module corrects the artificial intelligence model according to the correction data to generate a corrected artificial intelligence model. When the electronic device receives medical image data, the electronic device provides the medical image data to the cloud server, and the corrected artificial intelligence model analyzes the medical image data to generate analysis result data.

To sum up, in the medical image analysis method applying machine learning and system thereof, after the user inputs the medical image data through the electronic device, the electronic device provides the medical image data to the remote cloud server. As such, the medical image data is analyzed through the deep learning module and the artificial intelligence model built in the cloud server. In this way, the medical image data is effectively analyzed, and the analysis result data is generated.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a block diagram of a medical image analysis system according to an embodiment of the invention.

FIG. 2 is a schematic view of an analysis result of medical image data according to an embodiment of the invention.

FIG. 3 is a block diagram of a medical image analysis system according to another embodiment of the invention.

FIG. 4 is a schematic diagram of executing a medical image analysis system according to an embodiment of FIG. 3.

FIG. 5 is a flowchart of an initial phase according to an embodiment of FIG. 4.

FIG. 6 is a flowchart of a continuous correction phase according to the embodiment of FIG. 4.

FIG. 7 is a schematic diagram of continuous optimization performed on an artificial intelligence model according to an embodiment of the invention.

FIG. 8 is a flowchart of a medical image analysis method according to an embodiment of the invention.

### DESCRIPTION OF THE EMBODIMENTS

In order to make the invention more comprehensible, several embodiments are described below as examples of implementation of the invention. Moreover, elements/components/steps with the same reference numerals are used to represent the same or similar parts in the drawings and embodiments.

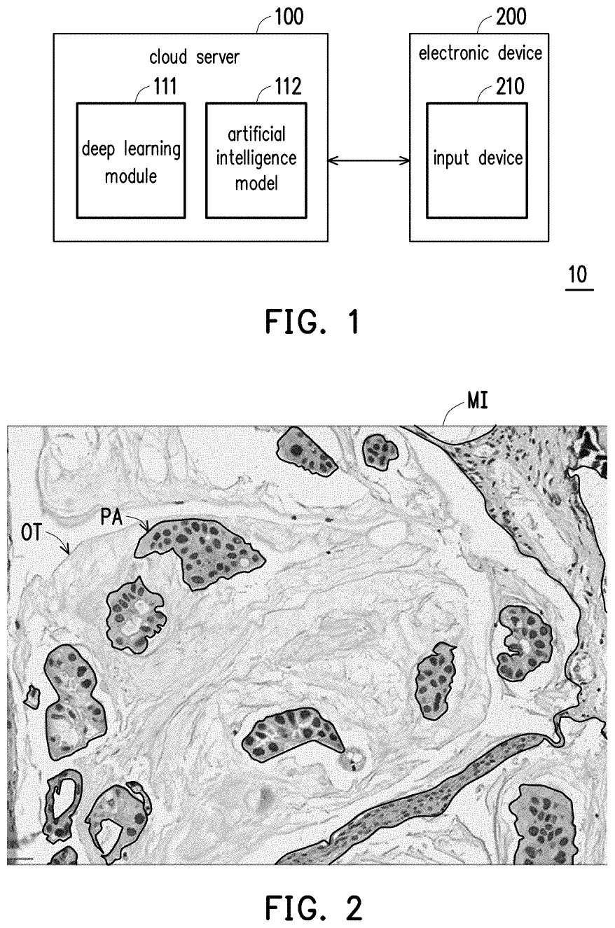

FIG. 1 is a block diagram of a medical image analysis system according to an embodiment of the invention. With reference to FIG. 1, a medical image analysis system 10 includes a cloud server 100 and an electronic device 200. The cloud server 100 stores a deep learning module 111 and an artificial intelligence (AI) model 112. The electronic device 200 includes an input device 210. In this embodiment, each of the cloud server 100 and the electronic device 200 may include a communication module, as such, wired or wireless data transmission can be performed between the cloud server 100 and the electronic device 200. The cloud server 100 may store a large volume of medical reference images for a user to build the required artificial intelligence model 112 by himself/herself. That is, the user may remotely connect to the cloud server 100 by operating on the electronic device 200. Moreover, the user may execute the deep learning module 111 and the artificial intelligence model 112 pre-built in the cloud server 100 through the communication module to perform a medical image analysis work. The artificial intelligence model 112 of this embodiment may perform automatic image identification and analysis on a medical image according to a pre-determined feature value or a determination condition. In other words, the electronic device 200 operated by the user is not required to be equipped with excessively high hardware performance, and the medical image analysis work can be conveniently performed through the remotely-built cloud server 100.

The electronic device 200 may be a computer device such as a desktop, a workstation, a laptop, or a tablet and the like. The electronic device 200 may communicate with the cloud server 100. The input device 210 may include a keyboard, a mouse, data input interfaces of various types or data transmission interfaces of various types and has a corresponding physical input circuit, piece of equipment, or hardware structure. The data input interfaces or the data transmission interfaces may be configured to transmit medical image data. For instance, the user may input a control command, correction data, training data, or the medical image data through the input device 210 of the electronic device 200 and provides the control command, the correction data, the training data, or the medical image data to the cloud server 100 to remotely control the cloud server 100.

In this embodiment, the user may input the correction data through the input device 210 of the electronic device 200. Furthermore, the electronic device 200 provides the correction data to the deep learning module 111 of the cloud server 100, as such, the deep learning module 111 corrects the artificial intelligence model 112 according to the correction data to generate a corrected artificial intelligence model, but the invention is not limited thereto. In an embodiment, the user may also directly input the correction data to the cloud server 100 through an input interface of the cloud server 100. In this embodiment, after the deep learning module 111 generates the corrected artificial intelligence model, the user may then input the medical image data (e.g., a section image of a body organ) through the electronic device 200. Furthermore, the electronic device 200 provides the medical image data to the cloud server 100 to analyze the medical image data through the corrected artificial intelligence model and generate analysis result data.

That is, in the medical image analysis system 10 in this embodiment, the user can perform the medical image analysis work through enabling the electronic device 200 to remotely communicate with the cloud server 100. Moreover, in this embodiment, the deep learning module 111 effectively corrects the artificial intelligence model 112 through the inputted correction data. Note that the correction data may be correspondingly generated according to a previous medical image analysis result, and the correction data may include, for example, a model parameter or a setting configured to correct the artificial intelligence model 112, which is not particularly limited by the invention. Hence, the medical image analysis system 10 of this embodiment is continuously optimized to correct the artificial intelligence model and provide an accurate medical image analysis function.

Note that the analysis result data generated by the cloud server 100 of this embodiment may be a quantitative analysis report, and the medical image data may come from medical image display equipment. The medical image data may include the medical image, and the medical image may be an immunohistochemistry (IHC) microscope image or other section images. In this embodiment, the medical image data may be, for example, a section image of a specific organ. Moreover, the medical image analysis system 10 of this embodiment is configured to analyze the medical image data by using the corrected artificial intelligence model, so as to effectively determine whether a cancer cell tissue exists in a section tissue of the specific organ in the section image.

FIG. 2 is a schematic view of an analysis result of medical image data according to an embodiment of the invention. With reference to FIG. 1 and FIG. 2, the electronic device 200 may provide the medical image data of a specific biological organ to the cloud server 100 to obtain the analysis result data, and the analysis result data refers to pathological information of the biological organ. For instance, FIG. 2 presents an analysis result of a medical image of a lung. The electronic device 200 may provide a section image MI of the lung to the cloud server 100. The artificial intelligence model 112 pre-built by the cloud server 100 may analyze the section image MI of the lung and generates an analysis result of the corresponding pathological information.

In FIG. 2, the section image MI includes a section tissue OT of the lung and a cancer cell tissue PA. The cloud server 100 may analyze information of each pixel in the section image MI to determine whether the cancer cell tissue PA exists in the section tissue OT in the section image MI of the lung through the artificial intelligence model 112. That is, the artificial intelligence model 112 can determine a location of a symptom or a sign of the cancer cell tissue PA to mark an area of the cancer cell tissue PA. In this embodiment, after the artificial intelligence model 112 completes the analysis, the cloud server 100 may transmit the analysis result back to the electronic device 200 in real time. That is, the medical image analysis system 10 of this embodiment can quickly obtain the analysis result of the section image MI.

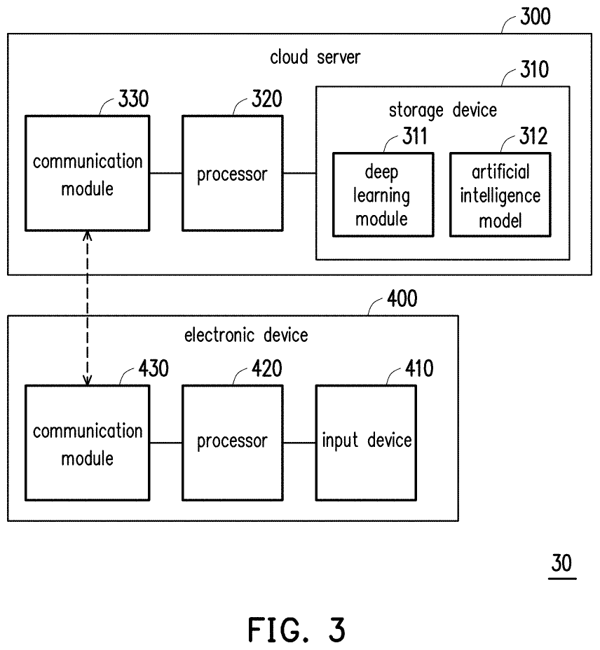

FIG. 3 is a block diagram of a medical image analysis system according to another embodiment of the invention. With reference to FIG. 3, a medical image analysis system 30 includes a cloud server 300 and an electronic device 400. The cloud server 300 may include a storage device 310, a processor 320, and a communication module 330. The electronic device 400 may include an input device 410, a processor 420, and a communication module 430. In this embodiment, the storage device 310 may store a deep learning module 311 and an artificial intelligence model 312. The user may process and analyze a large volume of data through the deep learning module 311 by using a large volume of medical reference images stored in the storage device 310 in advance to generate the artificial intelligence model 312 required by the user. In this embodiment, the cloud server 300 may be a file server, a database server, an application server, a workstation, a personal computer, or other similar types of computer devices with a computing capability. The electronic device 400 may be a computer device. The communication module 330 of the cloud server 300 may communicate with the communication module 430 of the electronic device 400, so that data transmission can be performed between the cloud server 300 and the electronic device 400.

The storage device 310 of the cloud server 300 may be a hard disk drive (HDD), a fixed or movable random access memory (RAM), a read-only memory (ROM), a flash memory, or similar devices in any form, or a combination of the foregoing devices. The storage device 310 may store the deep learning module 311 and the artificial intelligence model 312 and may also store one or a plurality of corrected artificial intelligence models generated by the deep learning module 311. Furthermore, the storage device 310 may also store various data, images, analysis results, etc. described in the embodiments of the invention.

Each of the processor 320 of the cloud server 300 and the processor 420 of the electronic device 400 may be a central processing unit (CPU), a microprocessor, a digital signal processor (DSP), a programmable controller, an application specific integrated circuit (ASIC), a system on chip (SoC) or other similar devices, or a combination of the foregoing devices.

Each of the communication module 330 of the cloud server 300 and the communication module 430 of the electronic device 400 may be a wired communication interface or a wireless communication interface. Communication may be performed, for example, through a cable in the wired communication module, and communication can be performed, for example, through Wi-Fi in the wireless communication module, but the invention is not limited thereto. For instance, the user may operate on the electronic device 400, so that the electronic device 400 can be connected to a network base station through Wi-Fi connection through the communication module 430 of the electronic device 400, and the electronic device 400 is then further connected to the communication module 330 of the cloud server 300 through the network base station in a wired or wireless manner. That is, remote connection to the cloud server 300 can be performed through the electronic device 400 operated by the user, and the user may perform medical image analysis through the cloud server 300 to obtain the analysis result in real time.

In this embodiment, the processor 320 of the cloud server 300 can execute the deep learning module 311. The deep learning module 311 may include a fully convolutional network (FCN) module to perform neural network operation on each of the medical reference images in the training data through the fully convolutional network module to build the artificial intelligence model. It is worth noting the fully convolutional network module in this embodiment may, for example, perform L-times upsampling operation of the K-times max pooling at the end of the executed neural network operation, where K and L are positive integers greater than 0, respectively.

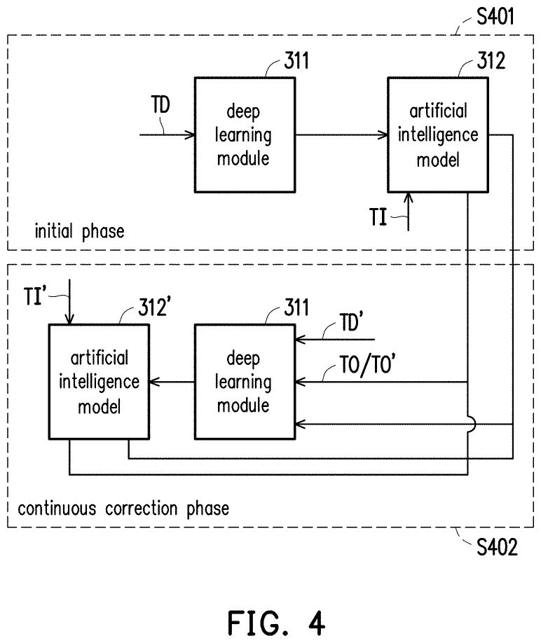

FIG. 4 is a schematic diagram of executing a medical image analysis system according to the embodiment of FIG. 3. With reference to FIG. 3 and FIG. 4, a medical image analysis system 30 of this embodiment may be operated in two phases, namely an initial phase S401 and a continuous correction phase S402. In the initial phase S401, first, a server builder may input training data TD to the cloud server 300 so as to execute the deep learning module 311 through the processor 320 according to the training data TD and generates the artificial intelligence model 312. The training data TD may include a plurality of medical reference images. Next, the user may provide testing data TI through the input device 410 of the electronic device 400. The testing data TI is the medical image data. The electronic device 400 inputs the testing data TI to the cloud server 300 through the communication module 430 so as to execute the artificial intelligence model 312 through the processor 320 according to the testing data TI and generates corresponding testing result data. The testing result data is the analysis result of the medical image data. Finally, the processor 320 of the cloud server 300 may generate corresponding correction data TO according to the testing result data. Therefore, the processor 320 can execute the deep learning module 311 again according to the artificial intelligence model 312 and the correction data TO to generate a corrected artificial intelligence model 312′.

In the continuous correction phase S402, first, the processor 320 of the cloud server 300 can execute the deep learning module 311 again according to the artificial intelligence model 312 and the correction data TO to generate the corrected artificial intelligence model 312′. Alternatively, in an embodiment, the user may provide another training data TD′ to the cloud server 300 through the input device 410 of the electronic device 400 at the same time. The other training data TD′ may include another medical reference image. Hence, the processor 320 of the cloud server 300 can execute the deep learning module 311 again according to the artificial intelligence model 312, the correction data TO, and the other training data TD′ to generate the corrected artificial intelligence model 312′. Note that comparing to the training data TD, the other training data TD′ is a medical image corresponding to different organ types or corresponding to an identical organ type with different cancer symptoms or different cancer signs. In this way, the deep learning module 311 can quickly generate the artificial intelligence model 312′ which can be applied to different organ types, different cancer symptoms, or different cancer signs based on the original artificial intelligence model 312. Moreover, after the deep learning module 311 generates the new artificial intelligence model 312′, the original artificial intelligence model 312 is not to be deleted. The artificial intelligence models 312 and 312′ can be applied to the medical image analysis at the same time. In other words, the cloud server 300 of this embodiment can effectively build a large number of artificial intelligence models configured to analyze the medical images of various organ types, various cancer symptoms, or various cancer signs.

Next, the user may continue to provide another testing data TI′ through the input device 410 of the electronic device 400, and the other testing data TI′ may include another medical image data. The processor 320 of the cloud server 300 can execute the artificial intelligence model 312′ according to the other testing data TI′ and generates corresponding another testing result data. The other testing result data is the analysis result of the other medical image data. Finally, the processor 320 of the cloud server 300 can generate corresponding next correction data TO′ according to the testing result data. The processor 320 of the cloud server 300 can execute the deep learning module 311 once again according to the artificial intelligence model 312′ and the next correction data TO′ to generate a next corrected artificial intelligence model.

Alternatively, in an embodiment, the user may provide another training data TD′ to the cloud server 300 through the input device 410 of the electronic device 400 at the same time. The other training data TD′ may include another medical reference image. Hence, the processor 320 of the cloud server 300 can execute the deep learning module 311 again according to the artificial intelligence model 312, the next correction data TO′, and the other training data TD′ to generate the next corrected artificial intelligence model. That is, the medical image analysis system 30 of this embodiment can effectively generate numerous artificial intelligence models corresponding to different organ types or corresponding to a same organ type with different cancer symptoms or different cancer signs through enhanced learning and continuous optimization. Furthermore, these artificial intelligence models are capable of providing accurate analysis results.

In addition, the correction data TO (or the correction data TO′) of this embodiment can be used to directly correct an error in the artificial intelligence model 312 (or in corrected the artificial intelligence model 312′). Therefore, when the processor 320 of the cloud server 300 corrects the artificial intelligence model 312 (or the corrected artificial intelligence model 312′) according to the correction data TO (or the correction data TO′) and another training data TD′ at the same time, a weight of the correction data TO (or the correction data TO′) is greater than a weight of the other training data TD′. That is, the importance of the correction data TO (or the correction data TO′) is greater in the medical image analysis system 30 of this embodiment.

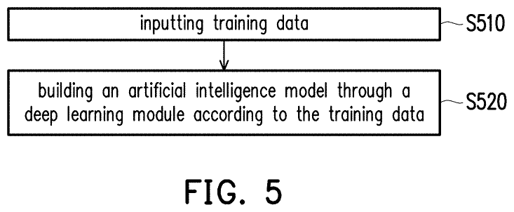

FIG. 5 is a flowchart of the initial phase S401 according to the embodiment of FIG. 4. With reference to FIG. 3 to FIG. 5, the steps of FIG. 5 may be applied to the medical image analysis system 30 of FIG. 3. In step S510, the user may input the training data TD to the cloud server 300 through the electronic device 400. In step S520, the processor 320 of the cloud server 300 may build the artificial intelligence model 312 through the deep learning module 311 according to the training data TD. That is, after the artificial intelligence model 312 of this embodiment is built, remote connection to the cloud server 300 may be established by the user through the electronic device 400 to analyze the medical image data through the artificial intelligence model 312 of the cloud server 300.

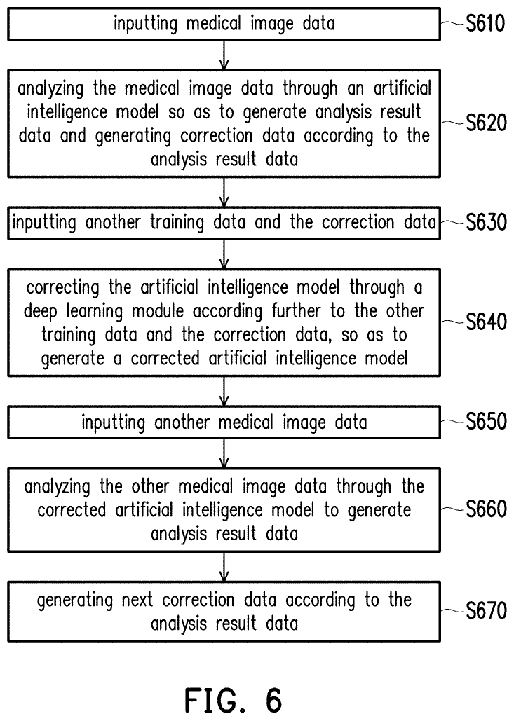

FIG. 6 is a flowchart of the continuous correction phase S402 according to the embodiment of FIG. 4. With reference to FIG. 3, FIG. 4, and FIG. 6, the steps of FIG. 6 may be applied to the medical image analysis system 30 of FIG. 3 and may be performed following step S520 of the embodiment of FIG. 5. In step S610, the user may input the testing data TI to the cloud server 300 through the electronic device 400. In step S620, the processor 320 of the cloud server 300 may analyze the testing data TI through the artificial intelligence model 312 to generate the analysis result data and generate the correction data TO according to the analysis result data. In step S630, the user may input the other training data TD′ and the correction data TO to the cloud server 300. In step S640, the processor 320 of the cloud server 300 may read and execute the deep learning module 311 to correct the artificial intelligence model 312 through the deep learning module 311 according further to the other training data TD′ and the correction data TO to generate the corrected artificial intelligence model 312′. In step S650, the user may input the medical image data (e.g., the testing data TI′ of FIG. 4) through the electronic device 400, and the electronic device 400 provides the medical image data to the cloud server 300 through the communication module 430. In step S660, the processor 320 of the cloud server 300 may analyze the medical image data through the corrected artificial intelligence model 312′ to generate the analysis result data. In step S670, the processor 320 of the cloud server 300 may generate the next correction data TO′ according to the analysis result data. In this embodiment, comparing to the training data TD, the other training data TD′ may include medical images of different organ types or may include medical images of the same type with different cancer symptoms or different cancer signs. In this way, the deep learning module 311 can quickly generate the artificial intelligence model 312′ which can be applied to different organ types, different symptoms, or different signs based on the original artificial intelligence model 312.

That is, after the artificial intelligence model 312 is built, remote connection to the cloud server 300 can be established by the user through the electronic device 400 to analyze the medical image data through the artificial intelligence model 312 of the cloud server 300. Moreover, the medical image data may include correct analysis result information obtained in advance. If an analysis result of the artificial intelligence model 312 is incorrect, the deep learning module 311 may correspondingly correct the artificial intelligence model 312 according to the correct analysis result information to generate the corrected artificial intelligence model 312′. Hence, by applying the medical image analysis system 30 of this embodiment, the situation in which the subsequent medical images cannot be correctly analyzed if the initially-built artificial intelligence model is an incorrect model can be effectively prevented. Moreover, in this embodiment, the medical image analysis system 30 may continuously correct the artificial intelligence model 312 through each of the analysis results. In this way, accuracy of the medical image analysis can be effectively increased, and the artificial intelligence model is not required to be re-built through additionally re-entering a large volume of training data. As such, computing resources of the cloud server 300 and the electronic device 400 are effectively saved.

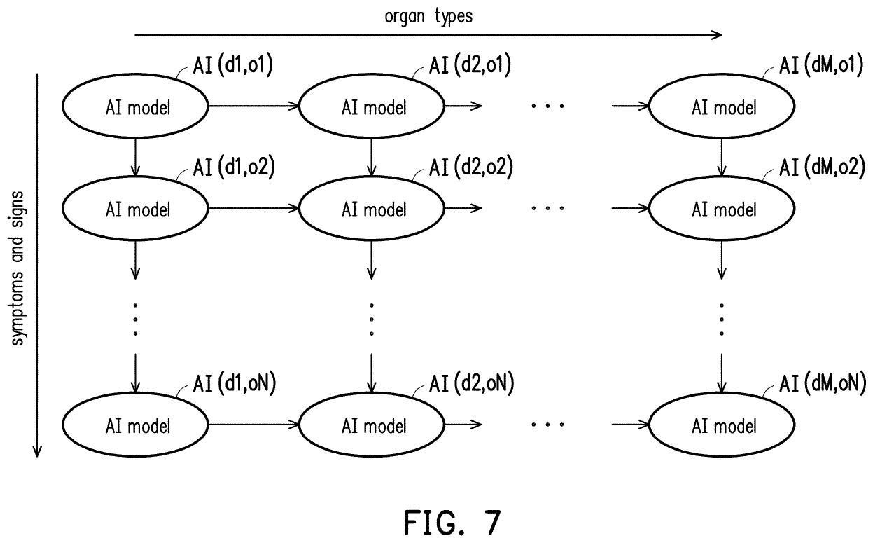

FIG. 7 is a schematic diagram of continuous optimization performed on an artificial intelligence model according to an embodiment of the invention. With reference to FIG. 3, FIG. 4, and FIG. 7, an artificial intelligence model AI(d1,o1) may be, for example, the artificial intelligence model 312 in the initial phase S401 of FIG. 4. The cloud server 300 may build the artificial intelligence model AI(d1,o1) first, and then the user can continuously input different pieces of medical image data (the testing data TI and the testing data TI′) and the testing result data (the correction data TO) to the cloud server 300 through the electronic device 400. In this way, the processor 320 of the cloud server 300 may perform enhanced learning through executing the deep learning module 311.

In this embodiment, when the pieces of medical image data, for example are pieces of medial image data corresponding to a same organ with different symptoms or different signs, the deep learning module 311 may generate numerous artificial intelligence models AI(d1,o2) to AI(d1,oN) corresponding to the same organ with different symptoms or different signs in sequence. N is a positive integer greater than 0. In this embodiment, when the pieces of medical image data, for example, are pieces of medical image data corresponding to different organ types, the deep learning module 311 can generate numerous artificial intelligence models AI(d2,o1) to AI(dM,o1) corresponding to the different organ types. M is a positive integer greater than 0. Similarly, the deep learning module 311 can further individually optimize the artificial intelligence models AI(d1,o2) to AI(d1,oN) to generate numerous artificial intelligence models AI(d2,o2) to AI(dM,oN) corresponding to different organ types with an identical symptom or an identical sign. In other words, the user can quickly build a required specific artificial intelligence model through the medical image analysis system 30 of this embodiment without re-entering a large volume of training data to re-generate the specific artificial intelligence model. Furthermore, the medical image analysis system 30 of this embodiment may continuously optimize the specific artificial intelligence model to generate an optimized artificial intelligence model.

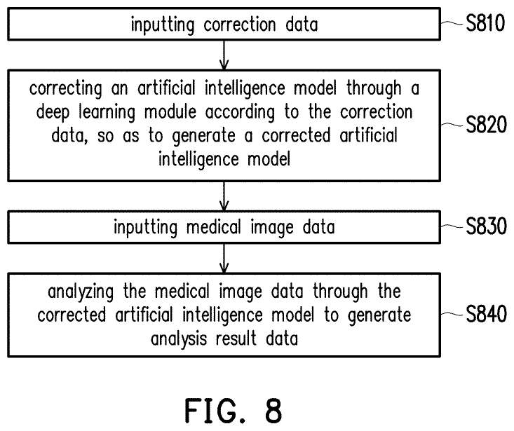

FIG. 8 is a flow chart of a medical image analysis method according to an embodiment of the invention. The medical image analysis method of FIG. 8 may at least be applied to the medical image analysis system 10 of FIG. 1 and the medical image analysis system 30 of FIG. 3. With reference to FIG. 1 and FIG. 8, in step S810, the electronic device 200 inputs the correction data to the cloud server 100. In step S820, the cloud server 100 corrects the artificial intelligence model 112 through the deep learning module 444 according to the correction data, so as to generate the corrected artificial intelligence model. In step S830, the electronic device 200 inputs the medical image data to the cloud server 100. In step S840, the cloud server 100 analyzes the medical image data through the corrected artificial intelligence model to generate the analysis result data. In other words, in the medical image analysis method of this embodiment, the correction data is continuously inputted to repeatedly correct the artificial intelligence model 112 to generate the optimized artificial intelligence model. Therefore, in the medical image analysis system 10 of this embodiment, highly accurate medical image analysis result is provided.

In addition, in this embodiment, people having ordinary skill in the art may acquire sufficient teachings, suggestions, and description of implementation of the medical image analysis system 10 with reference to the embodiments of FIG. 1 and FIG. 7, and that descriptions of other technical details and implementation are not further provided hereinafter.

In view of the foregoing, in the medical image analysis method and system thereof provided by the invention, a large volume of medical reference images can be stored in the cloud server remotely built in advance, as such, the user can build the required artificial intelligence model by himself/herself. Therefore, connection to the cloud server may be built by the user through the computer device, so the user can obtain the medical image analysis result in real time. Furthermore, in the medical image analysis method and system thereof provided by the invention, a large number of artificial intelligence models which can be applied to medical images of various organ types, various symptoms, or various signs can be generated through enhanced learning and continuous optimization, and the highly accurate medical image analysis result is also provided. Therefore, the medical image analysis method and system thereof provided by the invention can provide a convenient and effective medical image analysis function.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A medical image analysis method applying machine learning, adapted to a medical image analysis system, wherein the medical image analysis system comprises a cloud server and an electronic device, and the cloud server stores a deep learning module and an artificial intelligence model, wherein the medical image analysis method comprises:
inputting correction data to the deep learning module so that the deep learning module corrects the artificial intelligence model according to the correction data to generate a corrected artificial intelligence model; and
inputting medical image data to the electronic device, the electronic device providing the medical image data to the cloud server to analyze the medical image data through the corrected artificial intelligence model and generating analysis result data.

2. The medical image analysis method as claimed in claim 1, further comprising:
generating next correction data according to the analysis result data and inputting the next correction data to the deep learning module; and
correcting the corrected artificial intelligence model through the deep learning module according to the next correction data to generate a next corrected artificial intelligence model.

3. The medical image analysis method as claimed in claim 1, further comprising:
inputting training data to the deep learning module so that the deep learning module builds the artificial intelligence model according to the training data.

4. The medical image analysis method as claimed in claim 3, wherein the training data comprises a plurality of medical reference images, and the deep learning module comprises a fully convolutional network module, wherein the step of inputting the training data to the deep learning module so that the deep learning module builds the artificial intelligence model according to the training data comprises:
executing the fully convolutional network module through the deep learning module so that the fully convolutional network module performs neural network operation on each of the medical reference images to build the artificial intelligence model.

5. The medical image analysis method as claimed in claim 4, wherein the fully convolutional network module performs upsampling operation on each of the medical reference images.

6. The medical image analysis method as claimed in claim 1, further comprising:
inputting another medical image data to the electronic device, the electronic device providing the other medical image data to the cloud server to analyze the other medical image data through the artificial intelligence model and generating another analysis result data; and
generating the correction data according to the other analysis result data.

7. The medical image analysis method as claimed in claim 1, wherein the step of inputting the correction data to the deep learning module so that the deep learning module corrects the artificial intelligence model according to the correction data to generate the corrected artificial intelligence model comprises:
further inputting another training data to the deep learning module so that the deep learning module corrects the artificial intelligence model according to the correction data and the other training data to generate the corrected artificial intelligence model.

8. The medical image analysis method as claimed in claim 7, wherein a weight of the correction data is greater than a weight of the other training data.

9. The medical image analysis method as claimed in claim 7, wherein the other training data comprises another medical reference image.

10. A medical image analysis system applying machine learning, comprising:
a cloud server, storing a deep learning module and an artificial intelligence model; and
an electronic device, coupled to the cloud server,
wherein the deep learning module corrects the artificial intelligence model according to correction data when the cloud server receives the correction data to generate a corrected artificial intelligence model,
wherein the electronic device provides medical image data to the cloud server when the electronic device receives the medical image data, and the corrected artificial intelligence model analyzes the medical image data to generate analysis result data.
